(19) United States
(12) Patent Application Publication
(10) Pub. No.: US 2026/0308658 A1
(43) Pub. Date: Oct. 8, 2026

(54) Assay for Rapid Detection of Antibiotic Resistant Organisms

(71) Applicant: Queen's University at Kingston, Kingston (CA)

(72) Inventors: Mitchell Jeffs, Kingston (CA); Christopher Lohans, Kingston (CA)

(21) Appl. No.: 19/636,430

(22) Filed: Apr. 1, 2026

Related U.S. Application Data
(60) Provisional application No. 63/782,308, filed on Apr. 2, 2025.

(51) Int. Cl. C12Q 1/18; C12N 1/20; C12Q 1/37; C12R 1/19

(52) U.S. Cl. CPC C12Q 1/18; C12N 1/20; C12Q 1/37; C12R 2001/19; G01N 2333/952; G01N 2333/986

(57) ABSTRACT

In a method for detecting a carbapenemase-producing organism (CPO), a bacterial sample is lysed and the lysate is incubated with a carbapenem. The carbapenem is then transferred a solution including bacterial growth medium, an indicator bacterial strain that is susceptible to the carbapenem, a protease, and a cell viability indicator that can be metabolized by the indicator bacterial strain. After an incubation period, a change in the cell viability indicator confirms that the bacterial sample comprises a CPO, and no change in the cell viability indicator confirms that the bacterial sample does not include a CPO. A kit for an assay for detecting a CPO includes a lysis solution, a carbapenem, a bacterial growth medium, an indicator bacterial strain that is susceptible to the carbapenem, a protease, and a cell viability indicator that is metabolizable by the indicator bacterial strain.

### RELATED APPLICATION

This application claims the benefit of the filing date of Application No. 63/782,308 filed on Apr. 2, 2025, the contents of which are incorporated herein by reference in their entirety.

### FIELD

This invention relates to methods and tests for detecting antibiotic resistant organisms. More specifically, the invention provides low-cost assays and kits for rapid detection of carbapenemase-producing organisms without the need for inhibitors.

### BACKGROUND

Carbapenemases belong to the group of enzymes called β-lactamases, which inactivate β-lactam antibiotics via a hydrolytic mechanism. Carbapenemases are of particular concern, as unlike other β-lactamases (e.g., penicillinases, extended spectrum β-lactamases (ESBLs)), they are capable of hydrolyzing carbapenems, as well as other β-lactam antibiotics (e.g., penicillins, cephalosporins). Carbapenems are considered to be last-resort antibiotics usually reserved to treat infections caused by multi-drug resistant bacterial pathogens, where many other antimicrobials are ineffective. Thus, infections caused by carbapenemase-producing organisms (CPOs) are associated with increased patient mortality, and accordingly there is a need for low cost, rapid detection of CPOs in clinical settings to ensure timely and appropriate antimicrobial therapy and to prevent outbreaks in healthcare settings and long-term care facilities.

The most widely used phenotypic assay for CPO detection is the modified carbapenem inactivation method (mCIM; Pierce 2017). Although this assay is inexpensive and highly sensitive, it suffers from long turnaround times, requiring organism isolation (which typically takes 1-2 days) and an additional day to obtain a result from the test itself. Therefore, patients may be waiting for up to three days to obtain results that would guide the treatment of possibly life-threatening infections. Currently the only rapid phenotypic carbapenemase activity assay approved by the Clinical & Laboratory Standards Institute (CLSI) is the CARBA-NP test (Nordmann 2012). However, studies have reported that this test suffers from low sensitivity when applied to the detection of some weaker carbapenemases. Non-CLSI-approved resazurin-based methods have also been reported, but these have several limitations. For example, Teethaisong et al. (2016a) reported a resazurin chromogenic agar disk-diffusion method for the detection of CPOs. However, this assay requires seven hours to obtain results and the colourimetric readout on agar plates may be difficult to visually interpret. Additionally, this assay relies on the inhibition of carbapenemases for successful detection, with the inclusion of inhibitors such as ethylenediaminetetraacetic acid (EDTA) and phenylboronic acid (PBA). Several studies have reported that the use of EDTA for the detection of metallo-β-lactamases (a large subset of carbapenemases) is prone to high rates of false positives. Additionally, not all carbapenemases are susceptible to these inhibitors, which could negatively impact detection. Teethaisong et al. (2016b) also reported a microtitre plate assay employing resazurin for the detection of β-lactamase-producing bacteria. This test employs a series of serially diluted β-lactam antibiotics administered alongside different β-lactamase inhibitors. The assay then compares changes in resazurin metabolism by a test strain in each treatment. However, this assay is unsuitable for use in clinical settings due to the extensive preparatory work required, as every test requires an entire 96-well plate to be set up. A further limitation is that it relies on the inhibition of β-lactamase-producing strains by different β-lactamase inhibitors, rather than directly measuring β-lactam inactivation. Consequently, inhibitor-resistant β-lactamases will go undetected.

In addition to phenotypic tests, genotypic methods (e.g., molecular detection of carbapenemase-encoding genes by polymerase chain reaction (PCR)) have also been developed. However, these are often not suitable for lower resource sites due to the high costs associated with the instrumentation required and the need for highly skilled staff to run them. Additionally, novel carbapenemases or variants of known carbapenemases could be missed by these tests as PCR panels can only detect known enzymes.

It is evident that none of the currently available assays can fulfil all of what is needed for rapid and effective CPO detection, including short turnaround times, high sensitivity for all types of carbapenemases, and low cost.

### SUMMARY

According to one aspect of the invention there is provided a method for detecting a carbapenemase-producing organism (CPO), comprising: incubating a lysate of a bacterial sample with a carbapenem; transferring the carbapenem which was incubated with the sample lysate to a solution comprising bacterial growth medium, an indicator bacterial strain that is susceptible to the carbapenem, a protease, and a cell viability indicator that can be metabolized by the indicator bacterial strain; wherein a change in the cell viability indicator in the solution confirms that the bacterial sample comprises a CPO.

According to embodiments, the carbapenem may be selected from meropenem, ertapenem, and imipenem. In one embodiment, the carbapenem is imipenem.

In one embodiment, the carbapenem is in a stable preparation. In one embodiment, the carbapenem stable preparation comprises a disk.

In one embodiment, the bacterial growth medium comprises cation-adjusted Mueller Hinton broth (CAMHB).

In one embodiment, the indicator strain bacteria comprises an E. coli strain. In one embodiment, the E. coli strain comprises an ompA knockout strain.

In one embodiment, the cell viability indicator comprises resazurin.

In one embodiment, the protease comprises at least one of proteinase K, trypsin, chymotrypsin, and papain.

In one embodiment, the solution comprises at least one additive that increases metabolism of the cell viability indicator by the indicator strain bacteria. In one embodiment, the at least one additive comprises at least one of glucose and NADH.

In one embodiment, the method may comprise lysing the bacterial sample in a lysis solution containing at least one compound that maximizes hydrolysis of the carbapenem by the CPO. In one embodiment, the at least one compound comprises at least one of bicarbonate and zinc.

In one embodiment, the method may comprise adding a carbapenemase inhibitor to the lysate to differentiate between carbapenemase subclasses.

In one embodiment, the carbapenemase subclasses are selected from serine β-lactamases and metallo-β-lactamases.

According to another aspect of the invention there is provided a kit for an assay for detecting a carbapenemase-producing organism (CPO), comprising: a lysis solution; a carbapenem; a solution comprising bacterial growth medium; an indicator bacterial strain that is susceptible to the carbapenem; a protease; and a cell viability indicator that is metabolizable by the indicator bacterial strain.

According to embodiments, the carbapenem may be selected from meropenem, ertapenem, and imipenem. In one embodiment, the carbapenem is imipenem.

In one embodiment, the carbapenem is in a stable preparation. In one embodiment, the carbapenem stable preparation comprises a disk.

In one embodiment, the bacterial growth medium comprises cation-adjusted Mueller Hinton broth (CAMHB).

In one embodiment, the indicator bacterial strain comprises an E. coli strain. In one embodiment, the E. coli strain comprises an ompA knockout strain.

In one embodiment, the cell viability indicator comprises resazurin.

In one embodiment, the protease comprises at least one of proteinase K, trypsin, chymotrypsin, and papain.

In one embodiment, the lysis reagent comprises at least one additive selected from zinc and bicarbonate.

In one embodiment, the growth medium comprises at least one additive selected from glucose and NADH.

In one embodiment, the kit may comprise a carbapenemase inhibitor.

In one embodiment, the indicator bacterial strain comprises E. coli ΔompA; the carbapenem comprises imipenem; the protease comprises proteinase K; and the cell viability indicator comprises resazurin.

### BRIEF DESCRIPTION OF THE DRAWINGS

For a greater understanding of the invention, and to show more clearly how it may be carried into effect, embodiments will be described, by way of example, with reference to the accompanying drawings, wherein:

FIG. 1 is a flow diagram of a process for an assay according to a generalized embodiment.

FIGS. 2 and 3 are diagrams of processes for assays according to two exemplary embodiments.

### DETAILED DESCRIPTION OF EMBODIMENTS

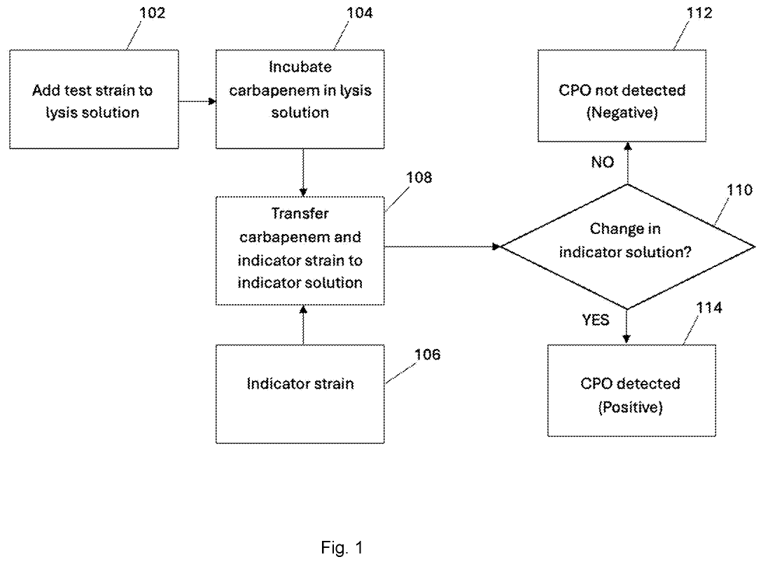

Described herein are assays for the rapid detection of carbapenemase-producing organisms (CPOs) which includes the subset of carbapenemase-producing Enterobacterales (CPE). According to embodiments, the assay couples carbapenem inactivation by a CPO to a cell viability reaction employing a bacterial indicator strain and a metabolizable cell viability indicator. A flow diagram showing steps of a generalized embodiment is shown in FIG. 1. Referring to FIG. 1, at 102 a CPO test strain is added to a lysis solution, and after lysis at 104 a carbapenem is incubated with the lysis solution. At 108, after incubation the carbapenem is transferred to an indicator solution for the cell viability reaction, with bacterial indicator strain also added to the indicator solution at 106. The bacterial indicator strain is susceptible to the carbapenem, and the cell viability reaction measures the viability of the bacterial indicator strain when it is exposed to the carbapenem that was incubated with the CPO test strain. At 110, after an incubation period the indicator solution is inspected for a detectable change. At 112, if the carbapenem was hydrolyzed by a CPO, the bacterial indicator strain will remain viable and thus metabolize the cell viability indicator, causing a detectable change indicating a CPO is present. If the test isolate is not a CPO, the carbapenem will remain active, thus killing the susceptible bacterial indicator strain. As a result, at 114 there will be minimal metabolism of the cell viability indicator and substantially no detectable change, indicating a CPO is not present.

Also described herein are kits for an assay for detecting a carbapenemase-producing organism (CPO). One embodiment may include a lysis solution, a carbapenem, a solution comprising bacterial growth medium, an indicator bacterial strain that is susceptible to the carbapenem, a protease, and a cell viability indicator that is metabolizable by the indicator bacterial strain.

Embodiments may include using a carbapenem such as, but not limited to, meropenem, ertapenem, and imipenem. In embodiments optimized for rapid detection of CPOs, imipenem may be used because it is hydrolyzed more quickly than other carbapenems by most carbapenemases and hence its use contributes to the rapidity of the assay. Accordingly, embodiments are described herein primarily with respect of imipenem, however, it will be appreciated that other carbapenems may be used.

Although not essential, embodiments may include using the carbapenem in a stabilized preparation, such as in a test disk. Test disks are typically made from of a small piece of paper or other porous material that has been supplemented with a known amount of carbapenem antibiotic; for example, a 10 μg imipenem disk may be used. Employing a disk advantageously avoids use of powdered or other forms of carbapenems which must be weighed out and dissolved in solvent prior to use, and which have a short shelf life once dissolved because of the instability of the drug to hydrolysis. Thus, in accordance with certain embodiments, the use of an imipenem disk in assays negates the need for preparing fresh imipenem solutions, and the disks are stable for long periods of time when stored between 8 and −20° C. Additionally, disks can be directly incubated in cell lysates to speed up the rate of reaction, resulting in faster hydrolysis of the carbapenem and faster CPO detection.

Embodiments may include using a cell lysis solution that maximizes hydrolysis of carbapenems by certain CPOs. The cell lysis solution may be optimized for a given carbapenem and/or CPO by including one or more compounds selected to maximize the hydrolysis of the carbapenem by the CPO. For example, bicarbonate may be added to increase the activity of OXA-type carbapenemases (e.g., OXA-48) and zinc may be added to increase the activity of metallo-β-lactamases (e.g., NDM, IMP, VIM).

Embodiments may include using a naturally occurring or engineered bacterial indicator strain that is selected due to its susceptibility or hyper-susceptibility to the carbapenem used. For example, a bacterial indicator strain may be a strain lacking one or more outer-membrane stabilizing proteins and/or having one or more altered outer-membrane stabilizing proteins, wherein the resulting membrane destabilization facilitates entry of one or more carbapenems. For example, the membrane protein(s) may be one or more of, but not limited to, OmpA, Braun's lipoprotein (Lpp), peptidoglycan-associated lipoprotein (Pal), and beta-barrel assembly machinery (Bam). In one embodiment the bacterial indicator strain may be a strain of Escherichia coli lacking the OmpA protein (i.e., a ΔompA strain). The inventors have previously shown that strains lacking this protein exhibit increased susceptibility to imipenem treatment compared to wild-type E. coli. This susceptibility allows for greater amounts of indicator strain to be used in the CPO detection assay without experiencing background colour change in CPO-negative samples due to residual cell viability. This increases the rate at which the cell viability indicator is metabolized in cases where the imipenem has been degraded by a CPO, which reduces assay turnaround time. It is expected that this strain would be susceptible to other carbapenems as well. In other embodiments, other E. coli strains or other bacterial strains that are susceptible to carbapenem antibiotics may be used as the indicator strains, wherein the indicator strain is selected for susceptibility to the specific carbapenem used.

Embodiments include using an indicator solution for the cell viability reaction with bacterial indicator strain, including a cell viability indicator that is metabolizable or otherwise affected by the bacterial indicator strain, whereby the cell viability indicator produces a detectable result indicating that the indicator strain is viable. The cell viability indicator may produce an observable result, such as a colour change. For example, in one embodiment resazurin is used as the cell viability indicator, wherein metabolism of resazurin by the indicator strain bacterium results in a colour change that is easily detected by observation. The indicator solution may include a protease, such as, but not limited to, one or more of proteinase K, trypsin, chymotrypsin, and papain.

According to one embodiment, the assay uses resazurin to determine the viability of an imipenem-susceptible bacterial strain once it has been exposed to imipenem that was incubated with a CPO test isolate, wherein a colour change indicates the imipenem susceptible bacterial strain is viable and hence a CPO is detected, and no colour change indicates the imipenem susceptible bacterial strain is not viable (i.e., it was killed by the imipenem) and hence no CPO is detected.

Prior approaches for detecting CPOs using resazurin typically work by detecting β-lactamases (or carbapenemases, more specifically) via inhibition tests (see, e.g., Nordmann, et al., 2021; Teethaisong, Y., et al., 2017; Teethaisong, Y., et al., 2016b). In general, according to such approaches, the viability of test strains in the presence of antibiotic alone is compared to that in the presence of antibiotic and β-lactamase inhibitor. However, the detection of CPOs based on carbapenemase inhibition presents a significant limitation to the prior approaches because they rely on the effectiveness of the inhibitor against the specific carbapenemase(s) produced by a CPO.

In contrast, embodiments described herein avoid the need for inhibitors to detect carbapenemases or CPOs by using a carbapenem-susceptible bacterial strain and a cell viability indicator directly as a marker for carbapenem hydrolysis. Thus, embodiments specifically detect carbapenemase activity, and may include one or more of (a) extraction of carbapenemase from a test isolate with a lysis solution that can be customized, (b) addition of a carbapenem (e.g., as a disk) to the lysate, which is then hydrolyzed by a carbapenemase in the sample, (c) transfer of the carbapenem disk to a suspension (which can be customized) of carbapenem hyper-susceptible indicator strain, which remains viable if the carbapenem has been degraded by a carbapenemase in the test strain, and (d) addition of cell viability indicator (e.g., resazurin) to measure the viability of the indicator strain in the presence of the carbapenem disk.

Some embodiments may provide a colourimetric assay for the clinical detection of carbapenemase-producing Enterobacterales (CPE) isolates that is rapid, does not require specialized equipment or highly trained technicians, and is simple to set up, thus making them suitable for implementation in clinical microbiology labs as a rapid, low-cost alternative to current CPE testing strategies. For example, in contrast to the mCIM, embodiments such as that described in Example 3 provide same-day results (e.g., within 3.5 h) without sacrificing sensitivity for carbapenemases with weaker hydrolytic activity (e.g., OXA-48-like).

One embodiment described in detail below (see Example 2) demonstrated high sensitivity (100%) and specificity (100%) and performed better than the CARBA-NP assay for detecting OXA-48-like producers, particularly those with low imipenem minimum inhibitory concentrations (MICs). Another embodiment (see Example 3) was tested in a pilot study by a clinical microbiology lab at Nova Scotia Health (Nova Scotia, Canada), demonstrating a sensitivity and specificity of 98% and 100%, respectively. In particular, embodiments do not rely on the use of β-lactamase inhibitors and thus overcome the limitations of prior approaches where detection of a carbapenemase is reliant on successful inhibition of the enzyme in the presence of a selected inhibitor. Embodiments do allow for β-lactamase inhibitors to be easily incorporated into the assay workflow to assess the inhibitor susceptibility profile of carbapenemase-producing Enterobacterales; however, detection of CPE is not reliant on successful carbapenemase inhibition. Embodiments may be optimized for direct detection of CPE from biological samples such as rectal swabs, thus negating the need for culturing the organisms overnight prior to testing, as is typically required for phenotypic CPE testing methods.

Embodiments are further described by way of the following non-limiting examples.

### Example 1

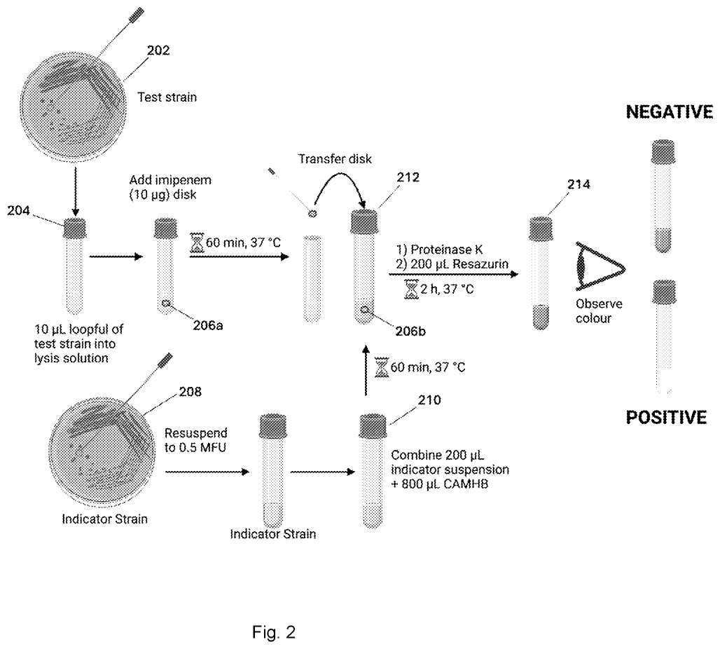

This example describes a process flow for a colourimetric assay according to one embodiment, with reference to FIG. 2.

Referring to FIG. 2, a bacterial test strain 202 (10 μL loop) is transferred to a lysis solution 204, and a carbapenem disk (10 μg imipenem) is added 206a. In a separate tube, a suspension of indicator strain 208 is prepared in cation adjusted Mueller Hinton Broth (CAMHB) 210 to approx. 0.5 McFarland Units (MFU), which is then diluted 5× in CAMHB for the assay (200 μL indicator suspension+800 μL CAMHB). Both the test strain lysate 206a and the diluted indicator strain 210 tubes are incubated at 37° C. for about 60 min. After this incubation, the imipenem disk 206b is transferred from the bacterial lysate to the indicator tube 212 which is followed by proteinase K (20 μL of a 500 μg/mL solution) and 200 μL of a 0.15 mg/mL solution of resazurin. This mixture is incubated at 37° C. for about 2 h, and is then monitored 214 for a colour change, wherein a colour change confirms that the bacterial sample comprises a CPO (positive) or no colour change confirms that the bacterial sample does not comprise a CPO (negative).

### Example 2

This example describes a process flow for a colourimetric assay according to one embodiment, similar to the protocol outlined in Example 1.

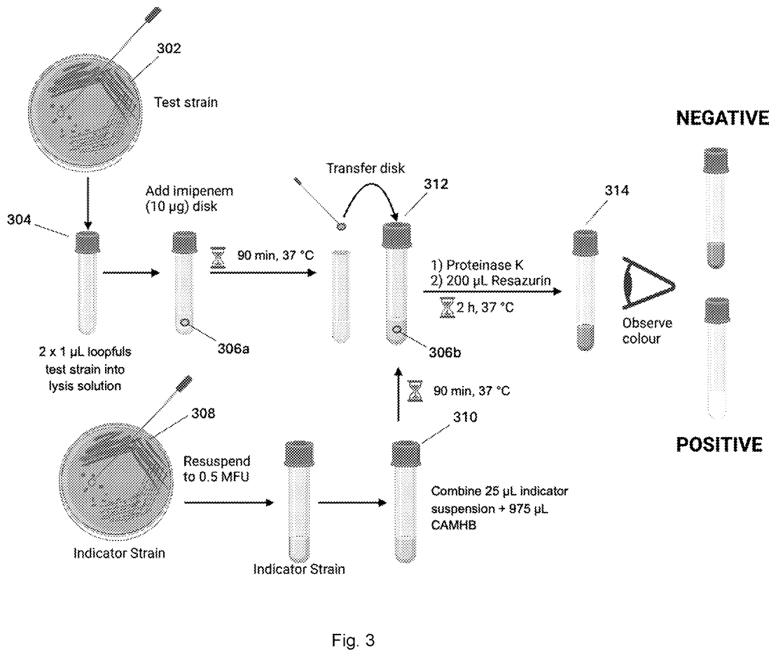

Referring to FIG. 3, a bacterial test strain 302 (2×1 μL loops) is transferred to a lysis solution 304, and a carbapenem disk (10 μg imipenem) is added 306a. In a separate tube, a suspension of indicator strain 308 is prepared in CAMHB 310 to approx. 0.5 MFU, which is then diluted 40× (25 μL indicator suspension+975 μL CAMHB). Both the test strain lysate 306a and the diluted indicator strain 310 tubes are incubated at 37° C. for about 90 min. After this incubation, the imipenem disk 306b is transferred from the bacterial lysate to the indicator tube 312, which is followed by proteinase K (20 μL of a 500 μg/mL solution) and 200 μL of a 0.15 mg/mL resazurin solution. This mixture is incubated at 37° C. for about 2 h, and is then monitored 314 for colour change, wherein a colour change confirms that the bacterial samples comprises a CPO (positive) or no colour change confirms that the bacterial sample does not comprise a CPO (negative).

### Example 3

This example describes in detail a colourimetric assay for the rapid detection of CPE strains, according to an embodiment wherein imipenem inactivation is coupled with resazurin as a cell viability indicator (referred to herein as the resaCPE assay). The resaCPE assay was performed on a panel of CPE clinical isolates, and for comparison the CARBA-NP test (Clinical and Laboratory Standards Institute 2024) was also performed on the same CPE clinical isolates.

### Materials

Imipenem disks were purchased from Hardy Diagnostics. BD BBL cation-adjusted Mueller-Hinton Broth (CAMHB), resazurin sodium salt, proteinase K and phenol red were purchased from Fisher Scientific. B-PER bacterial protein extraction reagent was purchased from Thermo Fisher Scientific.

### Bacterial Strains

E. coli BW25113 cells (National BioResource Project, NBRP) were transformed with pACYC184 (NBRP) vectors encoding representative carbapenemases (NDM-1, IMP-1, KPC-2, OXA-48) as previously reported (Jeffs 2023).

Carbapenemase-producing Enterobacterales (CPE) clinical isolates were collected by the clinical microbiology lab at Kingston General Hospital (KGH; Kingston, Canada) and ESBL-producing isolates were collected by Shared Hospital Laboratory (SHL; Toronto, Canada) during the normal course of care. The panel of isolates included 46 CPE strains and 29 ESBL-producers belonging to several Enterobacterales species. The CPE strains had been previously tested by the KGH microbiology lab for carbapenemase production via the mCIM test, and the presence of carbapenemase genes (NDM, KPC, OXA-48, VIM) was confirmed by multiplex PCR. The presence of ESBLs was verified by SHL via the ESBL double disk synergy test; all isolates were flagged negative for carbapenemase production based on meropenem and ertapenem minimum inhibitory concentrations (MICs).

All bacterial strains used in this study were cultured for 16-20 h at 37° C. on CAMHB agar plates. The CPE-negative control (E. coli ATCC 25922) and indicator strain (E. coli BW25113 ΔompA; National BioResource Project, NBRP) were grown on agar plates lacking antibiotic. Clinical CPE isolates were cultured on agar plates supplemented with 1 μg/mL imipenem and ESBL-producing isolates on plates supplemented with 100 μg/mL ampicillin.

resaCPE Assay

Test isolates, the CPE negative control, and the indicator strain were cultured as described above. On the day of the experiment, two 5 mL culture tubes were used per control and test isolate. To the first tube (lysis tube), 200 μL of B-PER was added, while 975 μL of sterile CAMHB was added to the second (indicator tube). An additional 5 mL culture tube was prepared for the viability control, which also received 975 μL of CAMHB.

In a new 5 mL culture tube, a suspension of indicator strain (E. coli BW25113 ΔompA) was prepared to OD600=0.1 (approximately equivalent to 0.5 McFarland Units, MFU) in 3 mL of CAMHB. Once prepared, 25 μL of indicator suspension was transferred to each indicator tube and the viability control tube. To each respective lysate tube, 2×1 μL loopfuls of the CPE-negative control or test isolate was transferred into the 200 μL of B-PER. These tubes were vortexed for 10-15 seconds. To each lysate tube, a 10 μg imipenem disk was added using sterile forceps. The indicator, viability control, and lysate tubes were then incubated at 37° C. for 1.5 h without shaking.

After the incubation, 20 μL of a 500 μg/mL proteinase K solution was added to each indicator tube and the viability control. The imipenem disk was transferred from the lysate tubes to each corresponding indicator tube using a sterile inoculation loop, and the lysate tubes were discarded. Lastly, 200 μL of 0.15 mg/mL resazurin solution was added to each indicator tube and the viability control. All tubes were then incubated at 37° C. for 2 h, after which the colour of the solutions was visually inspected. A colour change from blue/dark purple to pink was interpreted as CPE-positive, while no colour change was CPE-negative.

resaCPE Assay Employing Avibactam

Test isolates (E. coli BW25113 cells transformed with pACYC184 vectors encoding for NDM-1, IMP-1, KPC-2 or OXA-48), the CPE-negative control, and the indicator strain were cultured as described above. On the day of the experiment, four 5 mL culture tubes were used per control and test isolate. To the first two tubes (lysis tubes), 200 μL of B-PER was added, while 975 μL of sterile CAMHB was added to the last two (indicator tubes). An additional 5 mL culture tube was obtained for the viability control, which also received 975 μL of CAMHB.

In a new culture tube, a suspension of indicator strain (E. coli BW25113 ΔompA) was prepared to OD600=0.1 (approximately equivalent to 0.5 McFarland Units, MFU) in 5 mL of CAMHB. Once prepared, 25 μL of indicator suspension was transferred to each indicator tube and the viability control tube. To each respective lysate tube, 2×1 μL loopfuls of the CPE-negative control or test strain were transferred to the 200 μL of B-PER. These tubes were vortexed for 10-15 seconds. To the first lysate tube for each strain, avibactam solution was added to a final concentration of 16 μg/mL, along with a 10 μg imipenem disk, which was added using sterile forceps. To the second lysate tube for each sample, only the imipenem disk was added. The indicator, viability control, and lysate tubes were then incubated at 37° C. for 1.5 h without shaking. After the incubation, 20 μL of 500 μg/mL proteinase K solution was added to each indicator tube and the viability control. The imipenem disks were transferred from the lysate tubes to each corresponding indicator tube using a sterile inoculation loop, and the lysate tubes were discarded. Lastly, 200 μL of a 0.15 mg/mL resazurin solution was added to each indicator tube and the viability control. All tubes were then incubated at 37° C. for 2 h, after which the colour of the solutions was visually inspected.

### CARBA-NP Assay

The CARBA-NP test was performed as outlined in the CLSI M100 Edition 34 document (Clinical and Laboratory Standards Institute 2024). CARBA-NP solution A was prepared by adding 2 mL of 0.5% phenol red and 180 μL of 10 mM zinc sulfate to 16.6 mL of sterile water, which was then adjusted to pH 7.8. This solution was stored at 4° C. until the following day. Test strains and a carbapenemase-negative control (E. coli ATCC 25922) were cultured as described above. CARBA-NP solution B was prepared on the day of the assay by dissolving imipenem in CARBA-NP solution A to a concentration of 3 mg/mL. A 1 μL loopful of each test strain was added to two separate 1.5 mL tubes containing 100 μL of B-PER and vortexed for 5-10 seconds. To the first set of tubes, 100 UL of solution A was added; to the second set of tubes, 100 μL of solution B was added. These mixtures were incubated at 37° C. for 2 h, and colour change was monitored after this incubation period.

### Minimum Inhibitory Concentration (MIC) Testing

All MIC testing was conducted according to CLSI guidelines (Clinical and Laboratory Standards Institute 2024). Test isolates were cultured as described above. Imipenem stocks were prepared in sterile water and serially diluted in CAMHB in clear sterile 96-well non-treated flat-bottom plates (Corning). Cell suspensions were prepared by suspending colonies in CAMHB to OD600=0.1 (approx. 1×10⁸ CFU/mL). These suspensions were diluted in CAMHB to approx. 5×10⁶ CFU/mL, and 20 UL of the diluted suspensions was added to 180 μL of each of the imipenem dilutions. Plates were incubated for 20 h at 37° C. without shaking and OD600 readings were collected using a Synergy LX plate reader (Agilent BioTek) to determine MICs.

### Results

The performance of the resaCPE assay was assessed with 46 clinical CPE isolates of Citrobacter freundii, Escherichia coli, Enterobacter cloacae, Klebsiella pneumoniae, Klebsiella oxytoca, and Morganella morganii producing a range of carbapenemases (KPC, NDM, VIM, and OXA-48-like). All CPE isolates (13/13 KPC, 12/12 NDM, 13/13 VIM, 7/7 OXA-48-like, 1/1 KPC+NDM) were successfully detected with the resaCPE assay, as indicated by a strong colour change from blue/dark purple to pink (Table 1). Thus, the sensitivity of the resaCPE assay was 100% when challenged with this CPE panel.

The performance of the resaCPE assay was compared to the CARBA-NP test, which yielded a sensitivity of 95.6% (44/46 positives) (Table 1). The CARBA-NP test failed to detect two E. coli strains producing OXA-48-like carbapenemases with low imipenem MICs (2 μg/mL). Both of these strains were successfully detected with the resaCPE assay.

TABLE 1
Imipenem MICs (μg/mL) and resaCPE
and CARBA-NP assay results.

| | | Imipenem | | |
|---|---|---|---|---|
| | | MIC | resaCPE | CARBA-NP |
| Organism | Carbapenemase | (μg/mL) | Result | Result |
| C. freundii | VIM | 16 | + | + |
| C. freundii | KPC | 8 | + | + |
| C. freundii | | 4 | + | + |
| C. freundii | | 8 | + | + |
| C. freundii | | 8 | + | + |
| C. freundii | KPC + NDM | 16 | + | + |
| E. coli | VIM | 8 | + | + |
| E. coli | NDM | 16 | + | + |
| E. coli | | 8 | + | + |
| E. coli | | 16 | + | + |
| E. coli | | >32 | + | + |
| E. coli | | 8 | + | + |
| E. coli | | 8 | + | + |
| E. coli | | >32 | + | + |
| E. coli | KPC | 2 | + | + |
| E. coli | | 4 | + | + |
| E. coli | | 8 | + | + |
| E. coli | OXA-48-like | 2 | + | − |
| E. coli | | 32 | + | + |
| E. coli | | 16 | + | + |
| E. coli | | 16 | + | + |
| E. coli | | 2 | + | − |
| E. coli | | 16 | + | + |
| E. coli | | 32 | + | + |
| E. cloacae | VIM | 32 | + | + |
| E. cloacae | | >32 | + | + |
| E. cloacae | | 16 | + | + |
| E. cloacae | | 32 | + | + |
| E. cloacae | | 16 | + | + |
| E. cloacae | | 4 | + | + |
| E. cloacae | | 4 | + | + |
| E. cloacae | | 16 | + | + |
| E. cloacae | | 16 | + | + |
| E. cloacae | NDM | 8 | + | + |
| E. cloacae | | 16 | + | + |
| E. cloacae | | >32 | + | + |
| K. pneumoniae | VIM | 4 | + | + |
| K. pneumoniae | NDM | 8 | + | + |
| K. pneumoniae | | >32 | + | + |
| K. pneumoniae | KPC | 8 | + | + |
| K. pneumoniae | | 4 | + | + |
| K. pneumoniae | | 4 | + | + |
| K. pneumoniae | | 8 | + | + |
| K. pneumoniae | | 4 | + | + |
| K. oxytoca | KPC | 32 | + | + |
| M. morganii | VIM | 8 | + | + |

A panel of 29 ESBL-producing Enterobacterales (E. coli, K. pneumoniae, K. oxytoca, Proteus mirabilis) isolates was used to assess the specificity of the resaCPE test. None of the tested isolates produced a colour change, thus indicating that no false positives were obtained (specificity=100%) (Table 2).

TABLE 2
Imipenem MICs (μg/mL) and resaCPE and CARBA-NP
assay results for a panel of non-CPE isolates.

| Organism | ESBL Class | resaCPE Result |
|---|---|---|
| E. coli | D | − |
| E. coli | C | − |
| E. coli | C | − |
| K. oxytoca | A | − |
| K. oxytoca | A | − |
| K. pneumoniae | A | − |
| E. coli | A | − |
| E. coli | A | − |
| E. coli | A + C | − |
| K. pneumoniae | C | − |
| E. coli | A + C | − |
| E. coli | A + C | − |
| K. pneumoniae | A + C | − |
| K. pneumoniae | A + C | − |
| P. mirabilis | A | − |
| P. mirabilis | A | − |
| K. oxytoca | A | − |
| K. oxytoca | D | − |
| P. mirabilis | A | − |
| E. coli | C | − |
| E. coli | C | − |
| E. coli | C | − |
| E. coli | C | − |
| K. pneumoniae | A | − |
| K. pneumoniae | A | − |
| K. pneumoniae | A | − |
| K. oxytoca | A | − |
| K. oxytoca | A | − |
| K. oxytoca | A | − |

β-lactamase inhibitors (e.g., avibactam, relebactam, vaborbactam) are often administered alongside β-lactam antibiotics, protecting them from β-lactamase-catalyzed degradation. However, carbapenemase variants which are less susceptible to inhibition have begun to emerge. Current rapid testing methods do not incorporate clinically used inhibitors into their workflow; instead, labs tend to rely on growth-based susceptibility testing. These tests suffer from similar limitations as the mCIM, requiring overnight incubation (18-24 h) before results can be interpreted. As resistance to these β-lactamase inhibitors increases in prevalence, there is a need for rapid CPO detection methods that incorporate the testing of inhibitor susceptibility. Additionally, β-lactamase inhibitors can be used to broadly classify carbapenemases (e.g., serine β-lactamases (SBLs) vs. metallo-β-lactamases (MBLs)); this information may be important for clinicians, as combination therapies such as ceftazidime-avibactam are generally effective against SBL-producing isolates, but not those which produce MBLs. Incorporation of avibactam (16 μg/mL) into the biosensor assay workflow allowed for the successful differentiation between carbapenemases which are inhibited by avibactam (KPC-2, OXA-48) and those which are not (NDM-1, IMP-1). This information may prove useful in guiding treatment options for infections caused by multi-drug or pan-drug-resistant CPOs where therapeutic options are limited.

All cited publications are incorporated herein by reference in their entirety.

### EQUIVALENTS

While the invention has been described with respect to illustrative embodiments thereof, it will be understood that various changes may be made to the embodiments without departing from the scope of the invention. Accordingly, the described embodiments are to be considered merely exemplary and the invention is not to be limited thereby.

### REFERENCES

Clinical and Laboratory Standards Institute. 2024. CLSI M100 Performance Standards for Antimicrobial Susceptibility Testing 34^{th} Edition.
Jeffs, M. A., et al. Development of a whole-cell biosensor for β-lactamase inhibitor discovery. Chem Commun 59:12707-12710; 2023.
Nordmann, P., et al. Rapid detection of carbapenemase-producing Enterobacteriaceae. Emerg Infect Dis 18:1503-1507, 2012.
Pierce, V. M., et al. Modified Carbapenem Inactivation Method for Phenotypic Detection of Carbapenemase Production among Enterobacteriaceae. J. Clin. Microbiol. 55:2321-2333; 2017.
Teethaisong, Y., et al. Phenotypic detection of AmpC 13-lactamases, extended-spectrum β-lactamases and metallo-β-lactamases in Enterobacteriaceae using a resazurin microtitre assay with inhibitor-based methods. J Med Microbiol. 10:1079-1087; 2016a.
Teethaisong, Y., et al. A combined disc method with resazurin agar plate assay for early phenotypic screening of KPC, MBL and OXA-48 carbapenemases among Enterobacteriaceae. J Appl Microbiol. 121:408-14; 2016b.
Teethaisong, Y., et al. The performance of a resazurin chromogenic agar plate with a combined disc method for rapid screening of extended-spectrum-β-lactamases, AmpC β-lactamases and co-β-lactamases in Enterobacteriaceae. Microbiol Immunol. 61 (8): 297-304; 2017.

1. A method for detecting a carbapenemase-producing organism (CPO), comprising:
incubating a lysate of a bacterial sample with a carbapenem;
transferring the carbapenem which was incubated with the bacterial sample lysate to a solution comprising bacterial growth medium, an indicator bacterial strain that is susceptible to the carbapenem, a protease, and a cell viability indicator that can be metabolized by the indicator bacterial strain;
wherein a change in the cell viability indicator in the solution confirms that the bacterial sample comprises a CPO.

2. The method of claim 1, wherein the carbapenem is selected from meropenem, ertapenem, and imipenem.

3. The method of claim 1, wherein the carbapenem is imipenem.

4. The method of claim 1, when the carbapenem is in a stable preparation.

5. The method of claim 4, wherein the carbapenem stable preparation comprises a disk.

6. The method of claim 1, wherein the bacterial growth medium comprises cation-adjusted Mueller Hinton broth (CAMHB).

7. The method of claim 1, wherein the indicator strain bacteria comprises an E. coli strain.

8. The method of claim 7, wherein the E. coli strain comprises an ompA knockout strain.

9. The method of claim 1, wherein the cell viability indicator comprises resazurin.

10. The method of claim 1, wherein the protease comprises at least one of proteinase K, trypsin, chymotrypsin, and papain.

11. The method of claim 1, wherein the solution comprises at least one additive that increases metabolism of the cell viability indicator by the indicator strain bacteria.

12. The method of claim 11, wherein the at least one additive comprises at least one of glucose and NADH.

13. The method of claim 1, comprising lysing the bacterial sample in a lysis solution containing at least one compound that maximizes hydrolysis of the carbapenem by the CPO.

14. The method of claim 13, wherein the at least one compound comprises at least one of bicarbonate and zinc.

15. The method of claim 1, comprising adding a carbapenemase inhibitor to the lysate to differentiate between carbapenemase subclasses.

16. The method of claim 15, wherein the carbapenemase subclasses are selected from serine β-lactamases and metallo-β-lactamases.

17. A kit for an assay for detecting a carbapenemase-producing organism (CPO), comprising:
a lysis solution;
a carbapenem;
a solution comprising bacterial growth medium;
an indicator bacterial strain that is susceptible to the carbapenem;
a protease; and
a cell viability indicator that is metabolizable by the indicator bacterial strain.

18. The kit of claim 17, wherein:
the indicator bacterial strain comprises E. coli ΔompA;
the carbapenem comprises imipenem;
the protease comprises proteinase K; and
the cell viability indicator comprises resazurin.

19. The kit of claim 17, wherein the lysis reagent comprises at least one additive selected from zinc and bicarbonate.

20. The kit of claim 17, wherein the growth medium comprises at least one additive selected from glucose and NADH.
